(19) United States
(12) Patent Application Publication
(10) Pub. No.: US 2021/0378990 A1
(43) Pub. Date: Dec. 9, 2021

(54) COMPOSITIONS CONTAINING CHLOPHEDIANOL AND MENTHOL

(71) Applicant: GM Pharmaceuticals, Inc., Arlington, TX (US)

(72) Inventor: Odes W. Mitchell, Arlington, TX (US)

(21) Appl. No.: 17/334,725

(22) Filed: May 29, 2021

Related U.S. Application Data
(60) Provisional application No. 63/035,688, filed on Jun. 6, 2020.

(51) Int. Cl. A61K 31/137; A61K 31/045; A61K 9/00

(52) U.S. Cl. CPC A61K 31/137; A61K 9/0056; A61K 31/045

(57) ABSTRACT

In various embodiments, the present disclosure pertains to a composition composed of chlophedianol and menthol, or method of use thereof, having a form of a chewable composition, a chewable gummy, a chewable tablet, a chewable candy, a soft chewable, a hard chewable, a tablet, a capsule, a quick dissolving tablet or capsule, a lozenge, an orally disintegrating tablet, and a liquid composition. In some embodiments, the composition is formulated to alleviate respiratory tract infection symptoms, symptoms cause by allergies, symptoms cause by the common cold, symptoms cause by acute bronchitis, symptoms cause by pneumonia, symptoms cause by pertussis, symptoms cause by influenza, symptoms cause by tuberculosis, post-infectious cough symptoms, dry cough symptoms, productive cough symptoms, non-productive cough symptoms, or coughing and coughing-related symptoms selected from the group consisting of throat soreness, muscle strains or soreness in the abdomen or throat, throat irritation, and lung irritation, or combinations thereof.

### CROSS-REFERENCE TO RELATED APPLICATIONS

This patent application claims priority from, and incorporates by reference the entire disclosure of, U.S. Provisional Patent Application No. 63/035,688 filed on Jun. 6, 2020.

### TECHNICAL FIELD

The present disclosure relates generally to compositions containing chlophedianol and more particularly, but not by way of limitation, to compositions containing chlophedianol and menthol.

### BACKGROUND

This section provides background information to facilitate a better understanding of the various aspects of the disclosure. It should be understood that the statements in this section of this document are to be read in this light, and not as admissions of prior art.

A cough is a sudden expulsion of air through breathing passages that can help clear the passages of fluids, irritants, foreign particles, and microbes. Frequent coughing typically indicates the presence of a disease, such as a respiratory tract infection. During and after respiratory tract infections, repeated coughing produces inflammation which can lead to irritation and discomfort in the throat, which in turn produces more coughing. Many people seek various treatments to alleviate coughing and cough-related symptoms. As such, the present disclosure seeks to provide for various compositions (e.g., chewable compositions, tablets, capsules, quick dissolving tablets/capsules, lozenges, orally disintegrating tablets, and liquid compositions) to alleviate coughing and cough-related symptoms, such as, but not limited to, throat soreness, muscle strains or soreness in the abdomen or throat, throat irritation, lung irritation, and combinations of the same and like.

### SUMMARY OF THE INVENTION

This summary is provided to introduce a selection of concepts that are further described below in the Detailed Description. This summary is not intended to identify key or essential features of the claimed subject matter, nor is it to be used as an aid in limiting the scope of the claimed subject matter.

In an embodiment, the present disclosure pertains to a composition having chlophedianol and menthol. In some embodiments, the composition has a form that includes, without limitation, a chewable composition, a chewable gummy, a chewable tablet, a chewable candy, a soft chewable, a hard chewable, a tablet, a capsule, a quick dissolving tablet or capsule, a lozenge, an orally disintegrating tablet, and a liquid composition. In some embodiments, the composition is formulated to alleviate at least one of respiratory tract infection symptoms, symptoms cause by allergies, symptoms cause by the common cold, symptoms cause by acute bronchitis, symptoms cause by pneumonia, symptoms cause by pertussis, symptoms cause by influenza, symptoms cause by tuberculosis, post-infectious cough symptoms, dry cough symptoms, productive cough symptoms, non-productive cough symptoms, or coughing and coughing-related symptoms, such as, but not limited to, throat soreness, muscle strains or soreness in the abdomen or throat, throat irritation, and lung irritation, or combinations thereof.

In some embodiments, the menthol is present in the composition in a range of 1 mg to 10 mg. In some embodiments, the menthol has a concentration in the composition in a range of 0.001% (w/v) to 0.025% (w/v). In some embodiments, the menthol is present in the composition at an amount of 9 mg and the chlophedianol is present in the composition at an amount of 25 mg. In some embodiments, the menthol has a concentration in the composition of 0.015% (w/v) and the chlophedianol is present in the composition at an amount of 25 mg per 15 mL of the composition. In some embodiments, the chlophedianol is present in the composition in a range of 12.5 mg to 125 mg. In some embodiments, the chlophedianol is present in the composition in a range of 37.5 mg to 112.5 mg. In some embodiments, the chlophedianol is present in the composition in a range of 50 mg to 100 mg. In some embodiments, the chlophedianol is present in the composition in a range of 62.5 mg to 87.5 mg. In some embodiments, the chlophedianol is present in the composition in a range of 25 mg to 75 mg.

In some embodiments, the composition further includes at least of an absorption-enhancing agent, a flavoring agent, a releasing agent, a sugar alcohol, a texturizing agent, and anti-caking agent, a bulking agent, an emulsifying agent, a thickening agent, a solubilizing agent, a sweetening agent, a buffering agent, a preservative, stevia, mannitol, sucralose, crospovidone, microcrystalline cellulose (MCC), silica (silica dioxide), flavor, peppermint flavor, stearic acid, magnesium stearate, or combinations thereof.

In a further embodiment, the present disclosure pertains to a method of alleviating coughing or coughing-related symptoms. In general, the method includes administering a composition to a subject. In some embodiments, the composition includes chlophedianol and menthol. In some embodiments, the composition has a form, including, without limitation, a chewable composition, a chewable gummy, a chewable tablet, a chewable candy, a soft chewable, a hard chewable, a tablet, a capsule, a quick dissolving tablet or capsule, a lozenge, an orally disintegrating tablet, and a liquid composition. In some embodiments, the composition if formulated to alleviate at least one of respiratory tract infection symptoms, symptoms cause by allergies, symptoms cause by the common cold, symptoms cause by acute bronchitis, symptoms cause by pneumonia, symptoms cause by pertussis, symptoms cause by influenza, symptoms cause by tuberculosis, post-infectious cough symptoms, dry cough symptoms, productive cough symptoms, non-productive cough symptoms, or coughing and coughing-related symptoms, such as, but not limited to, throat soreness, muscle strains or soreness in the abdomen or throat, throat irritation, and lung irritation, or combinations thereof.

In some embodiments, the menthol is present in the composition in a range of 1 mg to 10 mg. In some embodiments, the menthol has a concentration in the composition in a range of 0.001% (w/v) to 0.025% (w/v). In some embodiments, the menthol is present in the composition at an amount of 9 mg and the chlophedianol is present in the composition at an amount of 25 mg. In some embodiments, the menthol has a concentration in the composition of 0.015% (w/v) and the chlophedianol is present in the composition at an amount of 25 mg per 15 mL of the composition.

In some embodiments, the composition is administered one to four times daily. In some embodiments, the composition is administered one to two times daily. In some embodiments, the composition is administered three to four times daily. In some embodiments, the composition is administered more than four times daily.

### DETAILED DESCRIPTION

It is to be understood that the following disclosure provides many different embodiments, or examples, for implementing different features of various embodiments. Specific examples of components and arrangements are described below to simplify the disclosure. These are, of course, merely examples and are not intended to be limiting. The section headings used herein are for organizational purposes and are not to be construed as limiting the subject matter described.

Generally, a cough is a sudden expulsion of air through large breathing passages that can help clear the passages of fluids, irritants, foreign particles, and microbes. Coughing is typically identified as a protective reflex, and can be repetitive with the cough reflex generally following three phases: (1) an inhalation; (2) a forced exhalation against a closed glottis; and (3) a violent release of air from the lungs following opening of the glottis. An occasional cough is a normal healthy function of the body, and the throat and airways are equipped with nerves that sense irritants, and in response, dispel them through the cough reflex. The throat and lungs normally produce a small amount of mucus to keep airways moist and to provide a thin covering layer that works as a protective barrier against irritants and microbes that may be inhaled. Some infrequent coughing helps mobilize mucus and typically has no damaging effects on the body. Additionally, coughing allows for the rapid removal of any unwelcome particles accidentally or inadvertently inhaled.

There are several different types of coughs having distinct characteristics and can typically be used to identify underlying issues that might be causing the cough. For example, if a cough brings up phlegm or mucus it is generally referred to as a productive cough and could suggest, for example, a respiratory tract infection. In contrast, a cough that does not produce mucus is generally referred to as a dry, or nonproductive, cough.

As briefly discussed above, a cough can be the result of allergies or a respiratory tract infection, such as, for example, the common cold, acute bronchitis, pneumonia, pertussis, influenza, or tuberculosis. In the vast majority of cases, acute coughs, that is, coughs lasting shorter than three weeks, are due to the common cold or minor allergies. Coughing caused by a respiratory tract infection or allergies can benefit from the use of cough suppressing agents such as, for example, chlophedianol, and additionally cough suppressing agents combined with additional components, such as, for example, menthol, that can help dilate the bronchioles (smaller branches of the bronchial airways in the respiratory tract) to facilitate in breathing.

Generally, after a respiratory tract infection or allergies have cleared, a person may be left with a post-infectious cough. A post-infectious cough is typically a dry, non-productive, cough that produces no phlegm. Symptoms may include a tightness in the chest, and a tickle in the throat. Post-infectious coughs may often persist for weeks after an illness. The cause of the cough can be attributed to, for example, inflammation similar to that observed in repetitive stress disorders. The repetition of coughing produces inflammation which produces discomfort, which in turn produces more coughing. Post-infectious coughs typically do not respond as well to conventional cough treatments, and generally respond better to a combination of anti-inflammatories with anesthetic properties (e.g., menthol) to treat the inflammation and soreness and a cough suppressant (e.g., chlophedianol) to reduce frequency of the cough until inflammation and soreness clears.

Chlophedianol is a centrally acting cough suppressant used for the treatment of coughs. Chlophedianol is used to provide relief of acute cough due to minor throat and bronchial irritation occurring with colds, respiratory tract infections, or inhaled irritants. Chlophedianol works by suppressing the cough reflex by a direct effect on the cough center in the medulla of the brain. In addition to cough suppression, chlophedianol has local anesthetic effects, as well as anticholinergic action. As such, compositions including chlophedianol exhibit great potential for cough suppressing formulations.

In addition to chlophedianol, menthol, typically derived from the herbs peppermint, eucalyptus, and pennyroyal, is a compound made from herbal volatile oils and shows many health benefits advantageous to the aid in recovery of allergies and respiratory tract infections, such as, for example, the common cold, acute bronchitis, pneumonia, pertussis, influenza, or tuberculosis. Menthol has pain-relieving and anti-inflammatory actions, and as such, can be utilized in treating respiratory conditions and post-respiratory disease symptoms. The ability to combat respiratory conditions stems from the ability of menthol to dilate the bronchioles, the smaller branches of the bronchial airways in the respiratory tract. Additionally, menthol can be utilized in treating colds, influenza, and bronchitis due to the expectorant actions of menthol, which involves thinning and loosening mucous congestion. Blocked sinuses and congested airways are common symptoms of allergies and respiratory tract infections, and menthol can help relieve such symptoms naturally and provide relief for variant airway discomfort. Moreover, menthol has antibacterial properties and pain-relieving effects due to its counterirritant and anesthetic properties, both of which prove beneficial when treating allergies, respiratory tract infections, or post-infectious coughs.

As such, the combinations of chlophedianol and menthol show advantageous properties to relieve coughing and coughing-related symptoms, such as, but not limited to, throat soreness, muscle strains or soreness in the abdomen or throat, throat irritation, lung irritation, and combinations of the same and like, brought on by allergies or respiratory tract infections and can additionally provide relief from post-infectious coughs.

Most tablet-form compositions, such as, for example, cough suppressants, are recommended to be taken with food. Typically, this is because if the compositions are consumed alone, the various constituents may not digest as well, do not fully get absorbed into the body, and can cause nausea or heartburn. For this reason, many people, find chewable or liquid compositions much easier on the stomach, and are generally able to ingest the compositions at any time of the day. Additionally, several people have difficulty swallowing tablets or capsules or simply do not like the sensation of swallowing tablets or capsules. This is especially true for people that may have more specific needs or impairments, such as children or the elderly. In these instances, swallowing entire tablets or capsules can be difficult and sometimes impossible. To alleviate these issues, people generally seek out chewable or liquid forms of different compositions or various types of lozenges and orally disintegrating tablets.

In addition to being easier to consume, chewable or liquid compositions are generally more optimized for absorption. For example, chewable compositions allow for higher absorption of constituents due to the ability of the composition to be processed in a similar manner as food. The act of chewing exposes the compositions to saliva which contain digestive enzymes that break down the compositions. Typically, the longer a composition is chewed, the greater the opportunity that these enzymes have to deconstruct the composition. This can lead to faster absorption times in the body, and thus faster relief of symptoms, such as, for example, coughing or coughing-related symptoms.

Similar to chewable compositions, liquid compositions have faster absorption times, can provide for higher absorption optimization, and are easily digestible. Moreover, like chewable compositions, liquids share similar advantages over tablets and capsules in relation to swallowing, as certain people, especially the elderly and young children, tend to have more difficulty swallowing tablets or capsules. As liquid compositions have faster absorption times, in general, liquid compositions, similar to chewable compositions, provide for faster relief of symptoms relating to coughing. Additionally, the compositions of the present disclosure can be in the form of quick dissolving tablets/capsules, lozenges, and orally disintegrating tablets.

In view of the aforementioned, the present disclosure relates generally to compositions containing chlophedianol and menthol for cough suppression and general relief of symptoms related to allergies, respiratory tract infections, and associated symptoms such as, but not limited to, coughing, throat soreness, muscle strains or soreness in the abdomen or throat, throat irritation, lung irritation, and combinations thereof. In some embodiments, the composition is a chewable composition. In some embodiments, the compositions is a liquid composition. In some embodiments, the compositions is a tablet, a capsule, a quick dissolving tablet/capsule, a lozenge, an orally disintegrating tablet, or a liquid composition.

In some embodiments, the compositions of the present disclosure can have various ingredients. For instance, in some embodiments, the ingredients include, without limitation chlophedianol, menthol, and combinations thereof. In some embodiments, the ingredient is chlophedianol. In some embodiments, the ingredient is menthol. In a particular embodiment of the present disclosure, the ingredient is a combination of chlophedianol and menthol. In some embodiments, the ingredient is a naturally occurring menthol. In some embodiments, the naturally occurring menthol is the natural isomer of menthol. In some embodiments, the naturally occurring menthol is L-menthol.

In some embodiments, the compositions includes chlophedianol in an amount of about 12.5 mg. In some embodiments, the chlophedianol is present in an amount of about 25 mg. In some embodiments, the chlophedianol is present in an amount of about 37.5 mg. In some embodiments, the chlophedianol is present in an amount of about 50 mg. In some embodiments, the chlophedianol is present in an amount of about 62.5 mg. In some embodiments, the chlophedianol is present in an amount of about 75 mg. In some embodiments, the chlophedianol is present in an amount of about 87.5 mg. In some embodiments, the chlophedianol is present in an amount of about 100 mg. In some embodiments, the chlophedianol is present in an amount of about 112.5 mg. In some embodiments, the chlophedianol is present in an amount of about 125 mg.

In some embodiments, the chlophedianol is present in a range of approximately 12.5 mg to approximately 25 mg, such as to be administered every 6 to 8 hours. In some embodiments, the chlophedianol is present in an amount of approximately 12.5 mg, such as to be administered every 6 to 8 hours. In some embodiments, the chlophedianol is present in an amount of approximately 25 mg, such as to be administered every 6 to 8 hours. In some embodiments, the chlophedianol can be administered up to an amount of approximately 50 mg over a 24 hour period. In some embodiments, the chlophedianol can be administered up to an amount of approximately 100 mg over a 24 hour period. In some embodiments, the chlophedianol is present in a range to allow for administration for up to 12 hours. In some embodiments, the chlophedianol is present in a range to allow for administration for 12 hours. In some embodiments, the chlophedianol is present in a range to allow for administration for over 12 hours.

In some embodiments, the chlophedianol is present in a range of about 12.5 mg to about 125 mg. In some embodiments, the chlophedianol is present in a range of about 37.5 mg to about 112.5 mg. In some embodiments, the chlophedianol is present in a range of about 50 mg to about 100 mg. In some embodiments, the chlophedianol is present in a range of about 62.5 mg to about 87.5 mg. In some embodiments, the chlophedianol is present in a range of about 25 mg to about 75 mg.

In some embodiments, the chlophedianol is present in a range of about 0.05% (w/v) to about 0.25% (w/v). In some embodiments, the chlophedianol is present in a range of about 0.06% (w/v) to about 0.24% (w/v). In some embodiments, the chlophedianol is present in a range of about 0.07% (w/v) to about 0.23% (w/v). In some embodiments, the chlophedianol is present in a range of about 0.08% (w/v) to about 0.22% (w/v). In some embodiments, the chlophedianol is present in a range of about 0.09% (w/v) to about 0.21% (w/v). In some embodiments, the chlophedianol is present in a range of about 0.10% (w/v) to about 0.20% (w/v).

In some embodiments, the chlophedianol is present in an amount of about 0.167% (w/v). In some embodiments, the chlophedianol is present in an amount of about 0.05% (w/v). In some embodiments, the chlophedianol is present in an amount of about 0.10% (w/v). In some embodiments, the chlophedianol is present in an amount of about 0.15% (w/v). In some embodiments, the chlophedianol is present in an amount of about 0.20% (w/v). In some embodiments, the chlophedianol is present in an amount of about 0.25% (w/v). In some embodiments, the chlophedianol is present in an amount up to about 0.25% (w/v).

In some embodiments, the chlophedianol is in the form a salt. In some embodiments, the chlophedianol is in the form of a hydrochloride salt. In some embodiments, the chlophedianol is chlophedianol hydrochloride.

In some embodiments, the menthol is present in a range of about 1 mg to about 20 mg. In some embodiments, the menthol is present in a range of about 2 mg to about 15 mg. In some embodiments, the menthol is present in a range of about 3 mg to about 12 mg. In some embodiments, the menthol is present in a range of about 4 mg to about 10 mg. In some embodiments, the menthol is present in a range of about 5 mg to about 9 mg. In some embodiments, the menthol is present in a range of about 6 mg to about 8 mg.

In some embodiments, the menthol is present in an amount of about 1 mg. In some embodiments, the menthol is present in an amount of about 2 mg. In some embodiments, the menthol is present in an amount of about 3 mg. In some embodiments, the menthol is present in an amount of about 4 mg. In some embodiments, the menthol is present in an amount of about 5 mg. In some embodiments, the menthol is present in an amount of about 6 mg. In some embodiments, the menthol is present in an amount of about 7 mg. In some embodiments, the menthol is present in an amount of about 8 mg. In some embodiments, the menthol is present in an amount of about 9 mg. In some embodiments, the menthol is present in an amount of about 3.375 mg. In some embodiments, the menthol is present in an amount of about 4.5 mg. In some embodiments, the menthol is present in an amount of about 10 mg. In some embodiments, the menthol is present in an amount of up to about 20 mg. In some embodiments, the menthol is present in an amount less than 1 mg.

In some embodiments, the menthol is present in a range of about 0.001% (w/v) to about 0.025% (w/v). In some embodiments, the menthol is present in a range of about 0.0075% (w/v) to about 0.0225% (w/v). In some embodiments, the menthol is present in a range of about 0.002% (w/v) to about 0.024% (w/v). In some embodiments, the menthol is present in a range of about 0.003% (w/v) to about 0.023% (w/v). In some embodiments, the menthol is present in a range of about 0.004% (w/v) to about 0.022% (w/v). In some embodiments, the menthol is present in a range of about 0.005% (w/v) to about 0.021% (w/v). In some embodiments, the menthol is present in a range of about 0.01% (w/v) to about 0.020% (w/v).

In some embodiments, the menthol is present in an amount of about 0.015% (w/v). In some embodiments, the menthol is present in an amount of about 0.010% (w/v). In some embodiments, the menthol is present in an amount of about 0.011% (w/v). In some embodiments, the menthol is present in an amount of about 0.012% (w/v). In some embodiments, the menthol is present in an amount of about 0.013% (w/v). In some embodiments, the menthol is present in an amount of about 0.014% (w/v). In some embodiments, the menthol is present in an amount of about 0.015% (w/v). In some embodiments, the menthol is present in an amount of about 0.016% (w/v). In some embodiments, the menthol is present in an amount of about 0.017% (w/v). In some embodiments, the menthol is present in an amount of about 0.018% (w/v). In some embodiments, the menthol is present in an amount of about 0.019% (w/v). In some embodiments, the menthol is present in an amount of about 0.020% (w/v). In some embodiments, the menthol is present in an amount up to about 0.025% (w/v).

As set forth in more detail herein, the compositions of the present disclosure can further include various excipients. For example, in some embodiments, the excipients include, without limitation, absorption-enhancing agents, flavoring agents, releasing agents, sugar alcohols, texturizing agents, anti-caking agents, bulking agents, emulsifying agents, thickening agents, solubilizing agents, sweetening agents, buffering agents, preservatives, and combinations thereof.

In some embodiments, the excipients include, without limitation, crospovidone, flavor, magnesium stearate, mannitol, microcrystalline cellulose, silicon dioxide, stearic acid, stevia, sucralose, citric acid, sodium benzoate, sodium citrate, sucralose, glycerin, xanthan gum, sorbitol solution, propylene glycol, honey, elderberry extract, potassium sorbate, stevia, and monk fruit extract, and combinations thereof.

In an embodiment of the present disclosure, the excipients include crospovidone, flavor, magnesium stearate, mannitol, microcrystalline cellulose, silicon dioxide, stearic acid, stevia, and sucralose. In an additional embodiment of the present disclosure, the excipients include citric acid, sodium benzoate, sodium citrate, sucralose, glycerin, xanthan gum, flavors, sorbitol solution, propylene glycol, honey, elderberry extract, potassium sorbate, stevia, and monk fruit extract.

TABLE 1
Example Chewable Tablet.

| | | Amount (mg) |
|---|---|---|
| | | per 300 mg |
| No. | Active Ingredients: | Tablet |
| 1 | Chlophedianol Hydrochloride | 25.00 |
| 2 | Stevia | 13.00 |
| 3 | L-Menthol | 9.00 |
| 4 | Mannitol | 125.00 |
| 5 | Sucralose | 13.00 |
| 6 | Crospovidone | 65.00 |
| 7 | MCC 102 | 34.50 |
| 8 | Silica | 3.00 |
| 9 | Peppermint flavor | 3.50 |
| 10 | Stearic Acid | 8.00 |
| 11 | Magnesium Stearate | 1.00 |

In a particular embodiment, shown above in Table 1, the present disclosure pertains to a chewable composition to alleviate and relieve coughing and coughing-related symptoms. In some embodiments, the chewable composition includes chlophedianol hydrochloride in amount of about 25 mg and 9 mg L-menthol. In some embodiments, the chewable compositions further includes excipients. In some embodiments, the excipients include, without limitation, stevia, mannitol, sucralose, crospovidone, microcrystalline cellulose (MCC; e.g., MCC 102), silica (i.e. silica dioxide), flavor (e.g., peppermint flavor), stearic acid, and magnesium stearate.

In some embodiments, the stevia can be in a range of about 1 mg to about 20 mg. In some embodiments, the mannitol can be in a range of about 50 mg to about 250 mg. In some embodiments, the sucralose can be in a range of about 10 mg to about 50 mg. In some embodiments, the crospovidone can be in a range of about 50 mg to about 150 mg. In some embodiments, the MCC can be in a range of about 25 mg to about 50 mg. In some embodiments, the silica can be in a range of about 1 mg to about 10 mg. In some embodiments, the peppermint flavor can be in a range of about 1 mg to about 10 mg. In some embodiments, the stearic acid can be in a range of about 1 mg to about 10 mg. In some embodiments, the magnesium stearate can be in a range of about 0.005 mg to about 2 mg.

In some embodiments, the chewable composition can be administered one to four times daily. In some embodiments, the chewable composition is administered once daily. In some embodiments, the chewable composition is administered twice daily. In some embodiments, the chewable composition is administered three times daily. In some embodiments, the chewable composition is administer four times daily. In some embodiments, the chewable composition is administered one or more times daily. In some embodiments, the chewable compositions is administered once every 12 hours. In some embodiments, the chewable composition is administered once every 12 to 24 hour.

In some embodiments, the chewable composition is administered as one chewable. In some embodiments, the chewable composition is administered as two chewables. In some embodiments, the chewable composition is administered as three or more chewables. In some embodiments, the chewable composition is administered as one or more chewables. In some embodiments, the chewable composition is in a form including, but not limited to, a chewable gummy, a chewable tablet, a chewable candy, a soft chewable, a hard chewable, a lozenge, an orally disintegrating tablets, quick dissolving tablets/capsules, and combinations thereof.

In some embodiments, the chewable composition of the present disclosure alleviates symptoms including, but not limited to, respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, and combinations thereof. In some embodiments, the chewable composition of the present disclosure alleviates coughing and coughing-related symptoms, such as, but not limited to, throat soreness, muscle strains or soreness in the abdomen or throat, throat irritation, lung irritation, and combinations thereof. In some embodiments, the chewable composition dilates bronchioles. In some embodiments, the chewable composition thins or loosens mucous congestion.

TABLE 2
Example Liquid Composition.

| No. | Ingredients: | W/V % |
|---|---|---|
| 1 | Chlophedianol Hydrochloride | 0.167 |
| | (25 mg in 15 mL) | |
| 2 | Stevia | 0.050 |
| 3 | L-Menthol | 0.015 |
| 4 | Sodium Benzoate | 0.100 |
| 5 | Sucralose | 0.030 |
| 6 | Xanthan Gum | 0.150 |
| 7 | Glycerin | 6.000 |
| 8 | Propylene Glycol | 5.000 |
| 9 | Flavor | 0.100 |
| 10 | Sorbitol Solution | 5.000 |
| 11 | Potassium Sorbate | 0.100 |
| 12 | Honey | 10.000 |
| 13 | Elderberry Extract | 0.500 |
| 14 | Sodium Citrate | 0.100 |
| 15 | Citric Acid | 0.100 |
| 16 | Monk Fruit Extract | 0.005 |
| 17 | Purified Water | QS to 100% |

In a particular embodiment, shown above in Table 2, the present disclosure pertains to a liquid composition to alleviate and relieve coughing and coughing-related symptoms. In some embodiments, the liquid composition includes chlophedianol hydrochloride in amount of about 25 mg in solution and L-menthol (e.g., 0.015% (w/v) as illustrated in Table 2). In some embodiments, the liquid composition includes chlophedianol hydrochloride in amount of about 12.5 mg in solution and L-menthol. In some embodiments, the liquid composition further includes excipients. In some embodiments, the excipients include stevia, sodium benzoate, sucralose, xanthan gum, glycerin, propylene glycol, flavor, sorbitol solution, potassium sorbate, honey, elderberry extract, sodium citrate, citric acid, monk fruit extract, and purified water.

In some embodiments, the stevia can be present in a range of about 0.01% (w/v) to 1% (w/v). In some embodiments, the sodium benzoate can be present in a range of about 0.05% (w/v) to 1.5% (w/v). In some embodiments, the sucralose can be present in a range of about 0.01% (w/v) to 1% (w/v). In some embodiments, the xanthan gum can be present in a range of about 0.1% (w/v) to 1% (w/v). In some embodiments, the glycerin can be present in a range of about 1% (w/v) to 10% (w/v). In some embodiments, the propylene glycol can be present in a range of about 1% (w/v) to 10% (w/v). In some embodiments, the flavor can be present in a range of about 0.01% (w/v) to 2% (w/v). In some embodiments, the sorbitol solution can be present in a range of about 1% (w/v) to 10% (w/v). In some embodiments, the potassium sorbate can be present in a range of about 0.01% (w/v) to 1% (w/v). In some embodiments, the honey can be present in a range of about 1% (w/v) to 15% (w/v). In some embodiments, the elderberry extract can be present in a range of about 0.01% (w/v) to 1.5% (w/v). In some embodiments, the sodium citrate can be present in a range of about 0.01% (w/v) to 1.5% (w/v). In some embodiments, the citric acid can be present in a range of about 0.01% (w/v) to 1.5% (w/v). In some embodiments, the monk fruit extract can be present in a range of about 0.001% (w/v) to 0.25% (w/v).

In some embodiments, the liquid composition can be administered one to four times daily. In some embodiments, the liquid composition is administered once daily. In some embodiments, the liquid composition is administered twice daily. In some embodiments, the liquid composition is administered three times daily. In some embodiments, the liquid composition is administer four times daily. In some embodiments, the liquid composition is administered one or more times daily. In some embodiments, the liquid composition is formulated to allow for administration for up to 12 hours. In some embodiments, the liquid composition is formulated to allow for administration for 12 hours. In some embodiments, the liquid composition is formulated to allow for administration for over 12 hours.

In some embodiments, the liquid composition is administered as one dose. In some embodiments, the liquid composition is administered as two doses. In some embodiments, the liquid composition is administered as three or more doses. In some embodiments, the liquid composition is administered as one or more doses. In some embodiments, the liquid composition is in a form including, but not limited to, a solution, a suspension, a syrup, an elixir, and combinations thereof.

In some embodiments, the liquid composition of the present disclosure alleviates symptoms including, but not limited to, respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, and combinations thereof. In some embodiments, the liquid composition of the present disclosure alleviates coughing and coughing-related symptoms, such as, but not limited to, throat soreness, muscle strains or soreness in the abdomen or throat, throat irritation, lung irritation, and combinations thereof. In some embodiments, the liquid composition dilates bronchioles. In some embodiments, the liquid composition thins or loosens mucous congestion.

In a further embodiment, the present disclosure pertains to a method of alleviating coughing or coughing-related symptoms. In some embodiments, the method includes administering a composition to a subject. In some embodiments, the composition includes chlophedianol and menthol. In some embodiments, the composition is at least one of a chewable compositions, a tablet, a capsule, a quick dissolving tablet/capsule, a lozenge, an orally disintegrating tablet, and a liquid composition. In some embodiments, the composition is at least one of a chewable composition and a liquid composition. In some embodiments, the composition alleviates at least one of respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, throat soreness, muscle strains or soreness in the abdomen or throat, throat irritation, lung irritation, and combinations thereof.

In some embodiments, the compositions of the present disclosure can be administered up to four times daily. In some embodiments, the composition is administered up to three times daily. In some embodiments, the composition is administered up to two times daily. In some embodiments, the composition is administered once daily. In some embodiments, the chlophedianol is present in a range of approximately 12.5 mg to approximately 25 mg, such as to be administered every 6 to 8 hours. In some embodiments, the chlophedianol is present in an amount of approximately 12.5 mg, such as to be administered every 6 to 8 hours. In some embodiments, the chlophedianol is present in an amount of approximately 25 mg, such as to be administered every 6 to 8 hours. In some embodiments, the chlophedianol can be administered up to an amount of approximately 50 mg over a 24 hour period. In some embodiments, the chlophedianol can be administered up to an amount of approximately 100 mg over a 24 hour period. In some embodiments, the chlophedianol is present in a range to allow for administration for up to 12 hours. In some embodiments, the chlophedianol is present in a range to allow for administration for 12 hours. In some embodiments, the chlophedianol is present in a range to allow for administration for over 12 hours. In various embodiments, in liquid compositions as disclosed herein, the chlophedianol can be expressed in, for example, mg per 25 mL of solution.

In view of the aforementioned, in an embodiment, an aspect of the present disclosure pertains to a chewable composition including chlophedianol and menthol. In some embodiments, the menthol is present in the composition in a range of 1 mg to 10 mg. In some embodiments, the menthol is present in an amount of 9 mg. In some embodiments, the chlophedianol is present in the composition in a range of 12.5 mg to 125 mg. In some embodiments, the chlophedianol is present in the composition in a range of 37.5 mg to 112.5 mg. In some embodiments, the chlophedianol is present in the composition in a range of 50 mg to 100 mg. In some embodiments, the chlophedianol is present in the composition in a range of 62.5 mg to 87.5 mg. In some embodiments, the chlophedianol is present in the composition in a range of 25 mg to 75 mg. In some embodiments, the chlophedianol is present in an amount of 12.5 mg. In some embodiments, the chlophedianol is present in an amount of 25 mg. In some embodiments, the chlophedianol is present in an amount of 37.5 mg. In some embodiments, the chlophedianol is present in an amount of 50 mg. In some embodiments, the chlophedianol is present in an amount of 62.5 mg. In some embodiments, the chlophedianol is present in an amount of 75 mg. In some embodiments, the chlophedianol is present in an amount of 87.5 mg. In some embodiments, the chlophedianol is present in an amount of 100 mg. In some embodiments, the chlophedianol is present in an amount of 112.5 mg. In some embodiments, the chlophedianol is present in an amount of 125 mg.

In some embodiments, the composition further includes at least one excipient, that can include, without limitation, absorption-enhancing agents, flavoring agents, releasing agents, sugar alcohols, texturizing agents, anti-caking agents, bulking agents, emulsifying agents, thickening agents, solubilizing agents, sweetening agents, buffering agents, preservatives, or combinations thereof. In some embodiments, the composition further includes at least one of stevia, mannitol, sucralose, crospovidone, microcrystalline cellulose (MCC), silica (silica dioxide), flavor, peppermint flavor, stearic acid, or magnesium stearate. In some embodiments, the composition is administered one or more times daily. In some embodiments, the composition is administered as one or more chewables. In some embodiments, the composition is in a form, that can include, without limitation a chewable composition, a chewable gummy, a chewable tablet, a chewable candy, a soft chewable, a hard chewable, a tablet, a capsule, a quick dissolving tablet or capsule, a lozenge, an orally disintegrating tablet, a liquid composition, and combinations thereof. In some embodiments, the composition is formulated to allow for administration for up to 12 hours. In some embodiments, the composition is formulated to allow for administration for 12 hours. In some embodiments, the composition is formulated to allow for administration for over 12 hours.

In some embodiments, the chewable composition alleviates at least one of respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, or combinations thereof. In some embodiments, the chewable composition alleviates coughing and coughing-related symptoms that can include, without limitation throat soreness, muscle strains or soreness in the abdomen or throat, throat irritation, lung irritation, and combinations thereof. In some embodiments, the chewable composition dilates bronchioles. In some embodiments, the chewable composition thins or loosens mucous congestion.

In an addition aspect, the present disclosure pertains to a liquid composition having chlophedianol and menthol. In some embodiments, the menthol is present in the composition in a range of about 0.001% (w/v) to about 0.025% (w/v). In some embodiments, the menthol is present in an amount of about 0.015% (w/v). In some embodiments, the chlophedianol is present in solution in a range of 12.5 mg to 125 mg. In some embodiments, the chlophedianol is present in solution in a range of 37.5 mg to 112.5 mg. In some embodiments, the chlophedianol is present in solution in a range of 50 mg to 100 mg. In some embodiments, the chlophedianol is present in solution in a range of 62.5 mg to 87.5 mg. In some embodiments, the chlophedianol is present in solution in a range of 25 mg to 75 mg. In some embodiments, the chlophedianol is present in solution in an amount of 12.5 mg. In various embodiments, in liquid compositions as disclosed herein, the chlophedianol can be expressed in, for example, mg per 25 mL of solution.

In some embodiments, the chlophedianol is present in solution in an amount of 25 mg. In some embodiments, the chlophedianol is present in solution in an amount of 37.5 mg. In some embodiments, the chlophedianol is present in solution in an amount of 50 mg. In some embodiments, the chlophedianol is present in solution in an amount of 62.5 mg. In some embodiments, the chlophedianol is present in solution in an amount of 75 mg. In some embodiments, the chlophedianol is present in solution in an amount of 87.5 mg. In some embodiments, the chlophedianol is present in solution in an amount of 100 mg. In some embodiments, the chlophedianol is present in solution in an amount of 112.5 mg. In some embodiments, the chlophedianol is present in solution in an amount of 125 mg. In various embodiments, in liquid compositions as disclosed herein, the chlophedianol can be expressed in, for example, mg per 25 mL of solution.

In some embodiments, the liquid composition further includes an excipient, including, but not limited to, absorption-enhancing agents, flavoring agents, releasing agents, sugar alcohols, texturizing agents, anti-caking agents, bulking agents, emulsifying agents, thickening agents, solubilizing agents, sweetening agents, buffering agents, preservatives, and combinations thereof. In some embodiments, the liquid composition further includes at least one of stevia, sodium benzoate, sucralose, xanthan gum, glycerin, propylene glycol, flavor, sorbitol solution, potassium sorbate, honey, elderberry extract, sodium citrate, citric acid, monk fruit extract, and purified water. In some embodiments, the composition is administered one or more times daily. In some embodiments, the composition is administered as one or more doses. In some embodiments, the liquid is in a form that can include, without limitation a solution, a suspension, a syrup, an elixir, and combinations thereof. In some embodiments, the liquid compositions alleviates at least one of respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, or combinations thereof. In some embodiments, the liquid composition alleviates coughing and coughing-related symptoms, such as, but not limited to throat soreness, muscle strains or soreness in the abdomen or throat, throat irritation, lung irritation, and combinations thereof. In some embodiments, the liquid composition dilates bronchioles. In some embodiments, the liquid composition thins or loosens mucous congestion.

In another aspect, the present disclosure pertains to a method of alleviating coughing or coughing-related symptoms. In some embodiments, the method includes administering a composition to a subject. In some embodiments, the composition includes chlophedianol and menthol, as disclosed herein. In some embodiments, the composition is at least one of a chewable composition and a liquid composition. In some embodiments, the composition alleviates at least one of respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, throat soreness, muscle strains or soreness in the abdomen or throat, throat irritation, lung irritation, or combinations thereof.

In some embodiments, the chlophedianol is present in an amount of 25 mg. In some embodiments, the administering is at least once a day. In some embodiments, the composition is a chewable composition. In some embodiments, the chewable composition is administered via one or more chewables. In some embodiments, the composition is a liquid composition. In some embodiments the liquid composition is administered via one or more doses. In some embodiments, the compositions is in a form, that includes, without limitation chewable compositions, tablets, capsules, quick dissolving tablets/capsules, lozenges, orally disintegrating tablets, liquid compositions, and combinations thereof. In various embodiments, in liquid compositions as disclosed herein, the chlophedianol can be expressed in, for example, mg per 25 mL of solution.

In an addition aspect, the present disclosure pertains a composition having chlophedianol and menthol. In some embodiments, the menthol is present in the composition in a range of 1 mg to 10 mg. In some embodiments, the menthol is present in an amount of 9 mg. In some embodiments, the chlophedianol is present in the composition in a range of 12.5 mg to 125 mg. In some embodiments, the chlophedianol is present in the composition in a range of 37.5 mg to 112.5 mg. In some embodiments, the chlophedianol is present in the composition in a range of 50 mg to 100 mg. In some embodiments, the chlophedianol is present in the composition in a range of 62.5 mg to 87.5 mg. In some embodiments, the chlophedianol is present in the composition in a range of 25 mg to 75 mg. In some embodiments, the chlophedianol is present in an amount of 12.5 mg. In some embodiments, the chlophedianol is present in an amount of 25 mg. In some embodiments, the chlophedianol is present in an amount of 37.5 mg. In some embodiments, the chlophedianol is present in an amount of 50 mg. In some embodiments, the chlophedianol is present in an amount of 62.5 mg. In some embodiments, the chlophedianol is present in an amount of 75 mg. In some embodiments, the chlophedianol is present in an amount of 87.5 mg. In some embodiments, the chlophedianol is present in an amount of 100 mg. In some embodiments, the chlophedianol is present in an amount of 112.5 mg. In some embodiments, the chlophedianol is present in an amount of 125 mg.

In some embodiments, the composition further includes at least one excipient, that can include, without limitation, absorption-enhancing agents, flavoring agents, releasing agents, sugar alcohols, texturizing agents, anti-caking agents, bulking agents, emulsifying agents, thickening agents, solubilizing agents, sweetening agents, buffering agents, preservatives, and combinations thereof. In some embodiments, the composition further includes at least one of stevia, mannitol, sucralose, crospovidone, microcrystalline cellulose (MCC), silica (silica dioxide), flavor, peppermint flavor, stearic acid, and magnesium stearate. In some embodiments, the composition is administered one or more times daily. In some embodiments, composition is administered as one or more chewables. In some embodiments, the composition is in a form, that can include, without limitation, a chewable composition, a chewable gummy, a chewable tablet, a chewable candy, a soft chewable, a hard chewable, a tablet, a capsule, a quick dissolving tablet or capsule, a lozenge, an orally disintegrating tablet, a liquid composition, and combinations thereof. In some embodiments, the compositions alleviates at least one of respiratory tract infection symptoms, symptoms cause by allergies, the common cold, acute bronchitis, pneumonia, pertussis, influenza, or tuberculosis, post-infectious cough symptoms, dry cough symptoms, productive cough symptoms, non-productive cough symptoms, or combinations thereof. In some embodiments, the composition alleviates coughing and coughing-related symptoms, such as, but not limited to throat soreness, muscle strains or soreness in the abdomen or throat, throat irritation, lung irritation, and combinations thereof. In some embodiments, the composition dilates bronchioles. In some embodiments, the composition thins or loosens mucous congestion. In some embodiments, the composition is formulated to allow for administration for up to 12 hours. In some embodiments, the composition is formulated to allow for administration for 12 hours. In some embodiments, the composition is formulated to allow for administration for over 12 hours.

Although various embodiments of the present disclosure have been described in the foregoing Detailed Description, it will be understood that the present disclosure is not limited to the embodiments disclosed herein, but is capable of numerous rearrangements, modifications, and substitutions without departing from the spirit of the disclosure as set forth herein.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

What is claimed is:

1. A composition comprising chlophedianol and menthol, wherein the composition has a form selected from the group consisting of a chewable composition, a chewable gummy, a chewable tablet, a chewable candy, a soft chewable, a hard chewable, a tablet, a capsule, a quick dissolving tablet or capsule, a lozenge, an orally disintegrating tablet, and a liquid composition; and
wherein the composition is formulated to alleviate at least one of respiratory tract infection symptoms, symptoms cause by allergies, symptoms cause by the common cold, symptoms cause by acute bronchitis, symptoms cause by pneumonia, symptoms cause by pertussis, symptoms cause by influenza, symptoms cause by tuberculosis, post-infectious cough symptoms, dry cough symptoms, productive cough symptoms, non-productive cough symptoms, or coughing and coughing-related symptoms selected from the group consisting of throat soreness, muscle strains or soreness in the abdomen or throat, throat irritation, and lung irritation, or combinations thereof.

2. The composition of claim 1, wherein the menthol is present in the composition in a range of 1 mg to 10 mg.

3. The composition of claim 1, wherein the menthol has a concentration in the composition in a range of 0.001% (w/v) to 0.025% (w/v).

4. The composition of claim 1, wherein the menthol is present in the composition at an amount of 9 mg and the chlophedianol is present in the composition at an amount of 25 mg.

5. The composition of claim 1, wherein the menthol has a concentration in the composition of 0.015% (w/v) and the chlophedianol is present in the composition at an amount of 25 mg per 15 mL of the composition.

6. The composition of claim 1, wherein the chlophedianol is present in the composition in a range of 12.5 mg to 125 mg.

7. The composition of claim 1, wherein the chlophedianol is present in the composition in a range of 37.5 mg to 112.5 mg.

8. The composition of claim 1, wherein the chlophedianol is present in the composition in a range of 50 mg to 100 mg.

9. The composition of claim 1, wherein the chlophedianol is present in the composition in a range of 62.5 mg to 87.5 mg.

10. The composition of claim 1, wherein the chlophedianol is present in the composition in a range of 25 mg to 75 mg.

11. The composition of claim 1, further comprising at least of an absorption-enhancing agent, a flavoring agent, a releasing agent, a sugar alcohol, a texturizing agent, and anti-caking agent, a bulking agent, an emulsifying agent, a thickening agent, a solubilizing agent, a sweetening agent, a buffering agent, a preservative, stevia, mannitol, sucralose, crospovidone, microcrystalline cellulose (MCC), silica (silica dioxide), flavor, peppermint flavor, stearic acid, magnesium stearate, or combinations thereof.

12. A method of alleviating coughing or coughing-related symptoms, the method comprising:
administering a composition to a subject;
wherein the composition comprises chlophedianol and menthol;
wherein the composition has a form selected from the group consisting of a chewable composition, a chewable gummy, a chewable tablet, a chewable candy, a soft chewable, a hard chewable, a tablet, a capsule, a quick dissolving tablet or capsule, a lozenge, an orally disintegrating tablet, and a liquid composition; and
wherein the composition if formulated to alleviate at least one of respiratory tract infection symptoms, symptoms cause by allergies, symptoms cause by the common cold, symptoms cause by acute bronchitis, symptoms cause by pneumonia, symptoms cause by pertussis, symptoms cause by influenza, symptoms cause by tuberculosis, post-infectious cough symptoms, dry cough symptoms, productive cough symptoms, non-productive cough symptoms, or coughing and coughing-related symptoms selected from the group consisting of throat soreness, muscle strains or soreness in the abdomen or throat, throat irritation, and lung irritation, or combinations thereof.

13. The method of claim 12, wherein the menthol is present in the composition in a range of 1 mg to 10 mg.

14. The method of claim 12, wherein the menthol has a concentration in the composition in a range of 0.001% (w/v) to 0.025% (w/v).

15. The method of claim 12, wherein the menthol is present in the composition at an amount of 9 mg and the chlophedianol is present in the composition at an amount of 25 mg.

16. The method of claim 12, wherein the menthol has a concentration in the composition of 0.015% (w/v) and the chlophedianol is present in the composition at an amount of 25 mg per 15 mL of the composition.

17. The method of claim 12, wherein the composition is administered one to four times daily.

18. The method of claim 12, wherein the composition is administered one to two times daily.

19. The method of claim 12, wherein the composition is administered three to four times daily.

20. The method of claim 12, wherein the composition is administered more than four times daily.
